(19) United States
(12) Patent Application Publication
(10) Pub. No.: US 2012/0272351 A1
(43) Pub. Date: Oct. 25, 2012

(54) SWEET PEPPER HYBRID 10BR46100

(76) Inventors: Jan de Bloois, Maasland (NL); Jacob Pieter Willem Haanstra, Dordrecht (NL)

(21) Appl. No.: 13/436,038

(22) Filed: Mar. 30, 2012

Related U.S. Application Data
(60) Provisional application No. 61/470,250, filed on Mar. 31, 2011.

(51) Int. Cl. A01H 5/00; A01H 5/06; A01H 5/04; A01H 5/10

(52) U.S. Cl. 800/260; 800/317.1; 435/410; 435/6.1

(57) ABSTRACT

The present invention relates to the field of plant breeding, particularly to the development of hybrid pepper variety 10BR46100. Pepper plants of the invention may exhibit a combination of traits including red colored blocky fruits, Tobamovirus resistance against Tobamovirus Pathotype 0 (P0) and Tobamovirus Pathotype 1-2 (P1-2), and resistance against Phytophthora capsici, representative seed of which have been deposited under NCIMB Accession No. 41824.

### CROSS-REFERENCE TO RELATED APPLICATIONS

This application claims priority to U.S. provisional patent application Ser. No. 61/470,250 filed Mar. 31, 2011.

The foregoing application and all documents cited in or during the prosecution of any foregoing applications(s) (“appln cited documents”) and all documents cited or referenced in the appln cited documents, and all documents cited or referenced herein (“herein cited documents”), and all documents cited or referenced in herein cited documents, together with any manufacturer's instructions, descriptions, products specifications, and product sheets for any products mentioned herein or in any document incorporated herein by reference, are hereby incorporated herein by reference, and may be employed in the practice of the invention.

### FIELD OF THE INVENTION

The present invention relates to the field of plant breeding and more particular to the development of hybrid pepper variety 10BR46100.

### BACKGROUND OF THE INVENTION

Sweet pepper plants of the species Capsicum annuum belong to the Nightshade family, also known as Solanaceae. It is an annual herbaceous flowering plant species native to South America.

Pepper plants are being cultivated worldwide for their highly nutritious fruits. In 2007 the acreage for sweet peppers in the United States was approximately 54.3 million, with a production of about 700,000 tons (source USDA). The pepper fruits have a high vitamin A and C content, as well as a high content in dietary fiber. They are also an excellent source of Calcium. Bell peppers are eaten raw, cooked, immature and mature and may be processed into powders, sauces, and salsas. The fruits in the unripe stage are usually green, but during ripening they usually become red, although other colors are known also such as: yellow, orange, purple, white, and brown.

There are various ways of cultivating peppers, the most common are: open field, greenhouse and shade house production. Although the species can be grown under a wide range of climatic conditions, it performs most successfully under dry and warm conditions.

Citation or identification of any document in this application is not an admission that such document is available as prior art to the present invention.

### BRIEF SUMMARY OF THE INVENTION

In one embodiment, the invention provides a pepper plant exhibiting a combination of traits including red colored blocky fruits, Tobamovirus resistance against Tobamovirus Pathotype 0 (P0) and Tobamovirus Pathotype 1-2 (P1-2), and resistance against Phytophthora capsici, representative seed of which have been deposited under NCIMB Accession No. 41824.

In one embodiment, the invention provides a pepper plant designated 10BR46100, representative seed of which have been deposited under NCIMB Accession No. 41824.

In one embodiment there is a plant grown from seeds, representative seed of which have been deposited under NCIMB Accession 41824.

In one embodiment the present invention relates to parts of a pepper plant of the invention, including parts of hybrid pepper variety 10BR46100, wherein the plant parts are involved in sexual reproduction, which include, without limitation, microspores, pollen, ovaries, ovules, embryo sacs or egg cells

In addition, the invention relates to parts of the plant that are suitable for vegetative reproduction, which include, without limitation, cuttings, roots, stems, cells, and protoplasts of the pepper plants of the invention.

In one embodiment the invention relates to a tissue culture of regenerable cells of the plant of the invention. Such a tissue culture can be derived, without limitation, from leaves, microspores, pollen, ovaries, ovules, embryo sacs or egg cells, cotyledon, hypocotyls, meristematic cells, roots, root tips, anthers, flowers, seeds and stems. The plants of the invention from which such parts can come from, include those of which representative seed been deposited under NCIMB No. 41824.

In one embodiment, there is provided progeny of hybrid pepper variety 10BR46100 produced by sexual or vegetative reproduction, grown from seeds, regenerated from the above-described plant parts, or regenerated from the above-described tissue culture of the hybrid pepper variety or a progeny plant thereof, representative seed of which have been deposited under NCIMB Accession No. NCIMB 41824.

In another embodiment, there is provided progeny of hybrid pepper variety 10BR46100 produced by sexual or vegetative reproduction, grown from seeds, regenerated from the above-described plant parts, or regenerated from the above-described tissue culture of the hybrid pepper variety or a progeny plant thereof, in which the regenerated plant exhibits a combination of traits including red colored blocky fruits, Tobamovirus resistance against Tobamovirus Pathotype 0 (P0) and Tobamovirus Pathotype 1-2 (P1-2), and resistance against Phytophthora capsici, representative seed of which has been deposited under NCIMB Accession No. NCIMB 41824.

Progeny of hybrid pepper variety 10BR46100 can be modified in one or more other characteristics, in which the modification is a result of, for example and without limitation, mutagenesis or transformation with a transgene.

In one embodiment, there is provided progeny of a hybrid pepper variety 10BR46100 produced by sexual or vegetative reproduction, grown from seeds, regenerated from the above-described plant parts, or regenerated from the above-described tissue culture of hybrid pepper variety or a progeny plant thereof, in which the regenerated plant exhibits a combination of traits including red colored blocky fruits, Tobamovirus resistance against Tobamovirus Pathotype 0 (P0) and Tobamovirus Pathotype 1-2 (P1-2), and resistance against Phytophthora capsici, and is modified in one or more other traits.

In another embodiment the invention relates to a method of producing an inbred pepper plant derived from a plant of the invention of which representative seed has been deposited under NCIMB Accession No. NCIMB 41824, comprising of the steps: a) preparing a progeny plant derived from hybrid pepper variety 10BR46100 by crossing the plant of a pepper plant exhibiting a combination of traits including red colored blocky fruits, Tobamovirus resistance against Tobamovirus Pathotype 0 (P0) and Tobamovirus Pathotype 1-2 (P1-2), and resistance against Phytophthora capsici, representative seed of which have been deposited under NCIMB Accession No. 41824 with a second pepper plant; b) crossing the progeny plant with itself or a second pepper plant to produce a seed of a progeny plant of a subsequent generation; c) growing a progeny plant of a subsequent generation from said seed and crossing the progeny plant of a subsequent generation with itself or a second pepper plant; and d) repeating step b) or c) for at least 1 more generation to produce an inbred pepper plant derived from the hybrid pepper variety 10BR46100.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims, terms such as “comprises”, “comprised”, and “comprising” and the like (e.g., “includes”, “included”, “including”, “contains”, “contained”, “containing”, “has”, “had”, “having”, etc.) can have the meaning ascribed to them in US Patent law, i.e., they are open ended terms. For example, any method that “comprises,” “has” or “includes” one or more steps is not limited to possessing only those one or more steps and also covers other unlisted steps. Similarly, any plant that “comprises,” “has” or “includes” one or more traits is not limited to possessing only those one or more traits and covers other unlisted traits. Similarly, the terms “consists essentially of” and “consisting essentially of” have the meaning ascribed to them in US Patent law, e.g., they allow for elements not explicitly recited, but exclude elements that are found in the prior art or that affect a basic or novel characteristic of the invention. See also MPEP § 2111.03. In addition, the term “about” is used to indicate that a value includes the standard deviation of error for the device or method being employed to determine the value.

These and other embodiments are disclosed or are obvious from and encompassed by the following Detailed Description.

Deposit

The Deposit with NCIMB Ltd, Ferguson Building, Craibstone Estate, Bucksburn, Aberdeen AB21 9YA, UK, under deposit accession number NCIMB 41824 was made pursuant to the terms of the Budapest Treaty. Upon issuance of a patent, all restrictions upon the deposit will be removed, and the deposit is intended to meet the requirements of 37 CFR §1.801-1.809. The deposit will be maintained in the depository for a period of 30 years, or 5 years after the last request, or for the effective life of the patent, whichever is longer, and will be replaced if necessary during that period.

### BRIEF DESCRIPTION OF THE DRAWING

The following detailed description, given by way of example, but not intended to limit the invention solely to the specific embodiments described, may best be understood in conjunction with the accompanying drawings, in which:

FIGS. 1A and 1B shows a technical questionnaire of the Dutch plant variety registration authorities (Board for Plant Varieties or Naktuinbouw)which relates to various embodiments of the invention.

### DETAILED DESCRIPTION OF THE INVENTION

The invention provides methods and compositions relating to plants, seeds and derivatives of a new hybrid pepper variety herein referred to as hybrid pepper variety 10BR46100. Hybrid pepper variety 10BR46100 is a commercial pepper hybrid distinct from other such hybrids.

The parents of hybrid pepper variety 10BR46100 were developed as follows: The mother is a dihaploid line, selected under Dutch glasshouse conditions from a group of dihaploid lines obtained in 2007 from a cross made in 2006 between two red blocky selection lines with group number 30416. The father of 10BR46100 is an inbred line, obtained by selection and inbreeding for at least nine times under Dutch glasshouse conditions, of a cross between two red blocky selection lines with group number 30252, of which inbreeding was started in 2003.

In one embodiment, a plant of the invention has all the morphological and physiological characteristics of hybrid pepper variety 10BR46100.

These physiological and morphological characteristics of the hybrid of the invention are summarized in table 1.

Table 2 summarizes the main differences between hybrid pepper variety 10BR46100 and the most similar variety 35-306. Embodiments of the invention advantageously have one or more, and most advantageously all, of these characteristics.

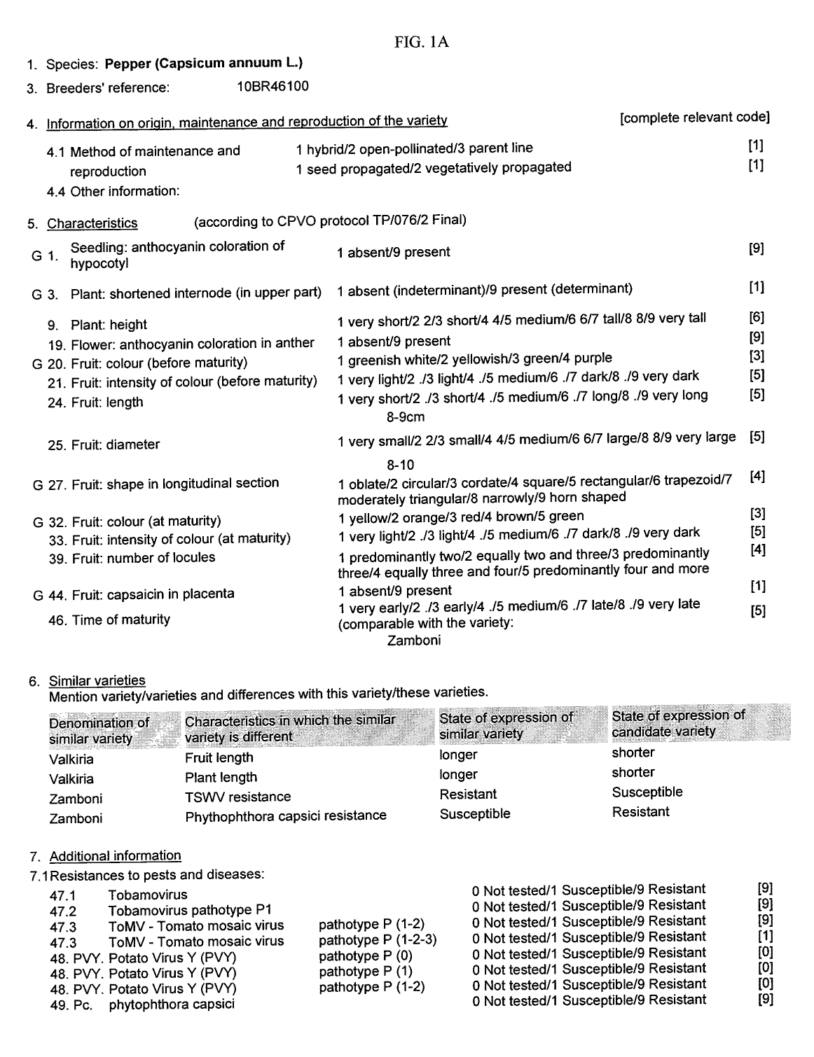

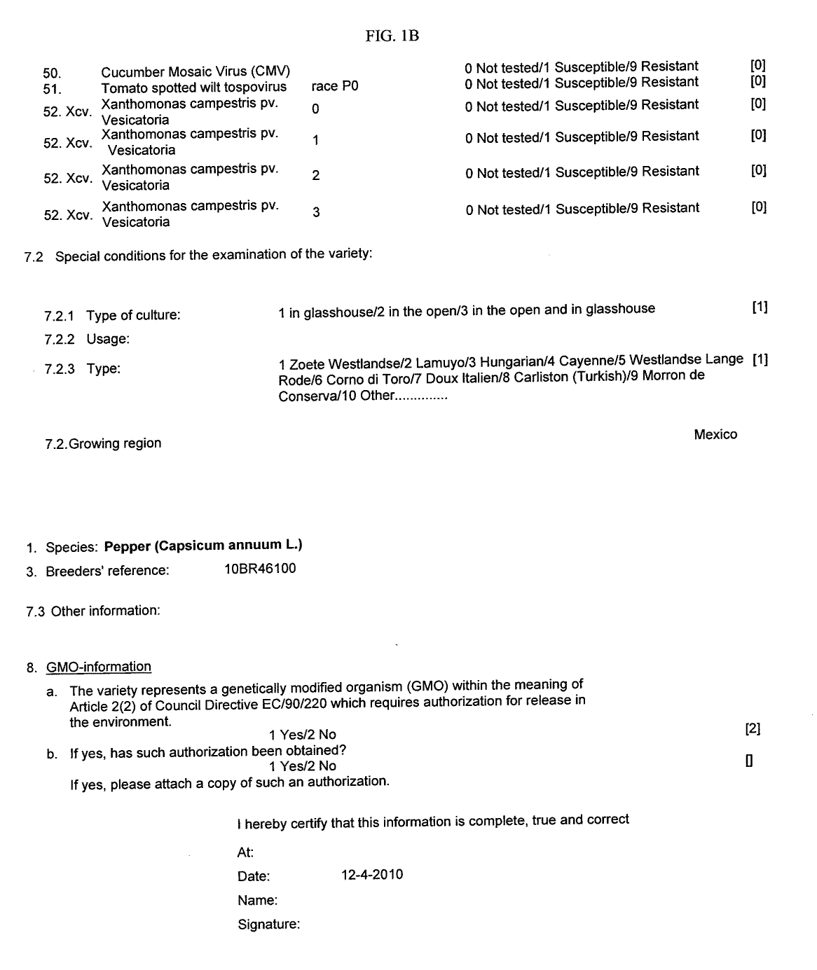

The information presented in tables 1 and 2 was determined in trial experiments in accordance with official Dutch plant variety registration authorities (Naktuinbouw). The terminology used in these tables is the official terminology as used by the Dutch plant variety registration authorities (Naktuinbouw) as of the filing date, and is thus clear for a person skilled in the art. Further information is described in FIGS. 1A and 1B.

TABLE 1
Physiological and morphological characteristics of hybrid
pepper variety 10BR46100.
Variety description information for 10BR46100

| | |
|---|---|
| General: | |
| Type: | Sweet bell pepper |
| Usage: | Fresh market |
| Type of culture: | Covered cultivation |
| Plant: | |
| Anthocyanin coloration of hypocotyls: | Present |
| Shortened internode (in upper part): | Absent |
| Height: | Medium to tall |
| Flower: anthocyanin coloration in anther | Present |
| Fruit: | |
| Color (before maturity): | Green |
| Intensity of color (before maturity): | Medium |
| Length: | Medium |
| Diameter: | Medium |
| Shape in longitudinal section: | Square |
| Color (at maturity): | Red |
| Intensity of colour (at maturity): | Medium |
| Number of locules: | Equally three and four |
| Capsaicin in placenta: | Absent |
| Time of maturity: | Medium |
| Disease and pest resistances: | |
| Tobamovirus (TMV) pathotype P0: | Resistant |
| Tobamovirus (TMV) pathotype P1: | Resistant |
| Tobamovirus (TMV) pathotype P1-2: | Resistant |
| Tobamovirus (TMV) pathotype P1-2-3: | Susceptible |
| Phytophthora capsici | Resistant |

TABLE 2
Comparison with closest known varieties Valkiria and Zamboni.

| | Characteristics in | State of | State of |
|---|---|---|---|
| Denomination of | which the similar | expression of | expression of |
| similar variety | variety is different | 35-306 | 10BR46100 |
| Valkiria | Fruit length | Longer | Shorter |
| Valkiria | Plant length | Longer | Shorter |
| Zamboni | TSWV resistance | Resistant | Susceptible |
| Zamboni | Phytophthora capsici | Susceptible | Resistant |
| | resistance | | |

There are numerous steps in the development of any novel, plant with desirable characteristics. Selection of traits is a very important aspect of plant breeding. Once desirable traits are identified, the plants with those desirable traits are crossed in order to recombine the desirable traits and through selection, varieties or parent lines are developed. The goal is to combine in a single variety or hybrid an improved combination of desirable traits from the parent plant. These important traits may include but are not limited to higher yield, field performance, fruit and agronomic quality such as fruit shape, color and length, resistance to diseases and insects, and tolerance to drought and heat.

Choice of breeding or selection methods depends on the mode of plant reproduction, the heritability of the trait(s) being improved, and the type of cultivar used commercially (e.g., F1 hybrid cultivar, pureline cultivar, etc.). Popular selection methods commonly include but are not limited to pedigree selection, modified pedigree selection, mass selection, and recurrent selection.

The complexity of inheritance influences choice of the breeding method. Backcross breeding is used to transfer one or a few favorable genes for a highly heritable trait into a desirable cultivar. This approach is used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

The development of commercial pepper cultivars relates to the development of pepper parental lines, the crossing of these lines, and the evaluation of the crosses. Pedigree breeding and recurrent selection breeding methods are used to develop cultivars from breeding populations. Breeding programs combine desirable traits from two or more varieties or various broad-based sources into breeding pools from which lines are developed by selfing and selection of desired phenotypes. The new lines are crossed with other lines and the hybrids from these crosses are evaluated to determine which have the desirable characteristics.

Pedigree breeding is used commonly for the improvement of self-pollinating crops or inbred lines of cross-pollinating crops. Two parents which possess favorable, complementary traits are crossed to produce an F1. An F2 population is produced by selfing one or several F1s or by intercrossing two F1s (sib mating). Selection of the best individuals is usually begun in the F2 population; then, beginning in the F3, the best individuals in the best families are selected. Replicated testing of families, or hybrid combinations involving individuals of these families, often follows in the F4 generation to improve the effectiveness of selection for traits with low heritability. At an advanced stage of inbreeding (i.e., F6 and F7), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding has been used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

Other methods of breeding may also relate to the single-seed descent procedure which refers to planting a segregating population, harvesting a sample of one seed per plant, and using the one-seed sample to plant the next generation. When the population has been advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the F2 plants originally sampled in the population will be represented by a progeny when generation advance is completed.

In addition to phenotypic observations, the genotype of a plant can also be examined. There are many laboratory-based techniques available for the analysis, comparison and characterization of plant genotype; these techniques include but are not limited to Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs—which are also referred to as Microsatellites), and Single Nucleotide Polymorphisms (SNPs)

Isozyme Electrophoresis and RFLPs have been widely used to determine genetic composition. Shoemaker and Olsen, (Molecular Linkage Map of Soybean (Glycine max) p 6.131-6.138 in S. J. O'Brien (ed) Genetic Maps: Locus Maps of Complex Genomes, Cold Spring Harbor Laboratory Press, Cold Spring Harbor, N.Y., (1993)) developed a molecular genetic linkage map that consisted of 25 linkage groups with about 365 RFLP, 11 RAPD, three classical markers and four isozyme loci. See also, Shoemaker, R. C., RFLP Map of Soybean, p 299-309, in Phillips, R. L. and Vasil, I. K., eds. DNA-Based Markers in Plants, Kluwer Academic Press, Dordrecht, the Netherlands (1994).

SSR technology is currently the most efficient and practical marker technology; more marker loci can be routinely used and more alleles per marker locus can be found using SSRs in comparison to RFLPs. For example, Diwan and Cregan described a highly polymorphic microsatellite locus in soybean with as many as 26 alleles. (Diwan, N. and Cregan, P. B., Theor. Appl. Genet. 95:22-225, 1997.) SNPs may also be used to identify the unique genetic composition of the invention and progeny varieties retaining that unique genetic composition. Various molecular marker techniques may be used in combination to enhance overall resolution.

Molecular markers, which include markers identified through the use of techniques such as Isozyme Electrophoresis, RFLPs, RAPDs, AP-PCR, DAF, SCARs, AFLPs, SSRs, and SNPs, may be used in plant breeding. One use of molecular markers is Quantitative Trait Loci (QTL) mapping. QTL mapping is the use of markers which are known to be closely linked to alleles that have measurable effects on a quantitative trait. Selection in the breeding process is based upon the accumulation of markers linked to the positive effecting alleles and/or the elimination of the markers linked to the negative effecting alleles from the plant's genome.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Molecular markers may also be used to identify and exclude certain sources of germplasm as parental varieties or ancestors of a plant by providing a means of tracking genetic profiles through crosses.

Mutation breeding is another method of introducing new traits into pepper varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

The production of double haploids may also be used for the development of homozygous lines in a breeding program. Double haploids are produced by the doubling of a set of chromosomes from a heterozygous plant to produce a completely homozygous individual. For example, see Wan et al., Theor. Appl. Genet., 77:889-892, 1989

The pepper plant of the invention may be arrived at through crossing of inbred lines or through selection of the disclosed desirable characteristics by any of the breeding the selection methods mentioned above.

Further useful traits can be introduced by backcrossing or useful traits can be introduced directly into the plant of the invention, being a plant of hybrid pepper variety 10BR46100, by genetic transformation techniques; and, such plants of inbred hybrid pepper variety 10BR46100 that have additional genetic information introduced into the genome or that express additional traits by having the DNA coding there for introduced into the genome via transformation techniques, are within the ambit of the invention, as well as uses of such plants, and the making of such plants.

In this regard, and in regard to the color traits of the peppers of plants of the invention, mention is made of Thorup et al., “Candidate gene analysis of organ pigmentation loci in the Solanaceae,” Proc. Natl. Acad. Sci. USA (2000) 97(21): 11192-11197. Carotenoids are the red, orange, and yellow molecules that act as photoprotective agents and accessory light-harvesting pigments, and add nutritional and ornamental value to plants, and enzymes in this pathway have been characterized and the structural genes have been cloned from a wide array of higher plants, including pepper (Capsicum annuum L.). The genetic basis of variation in fruit color due to alterations in carotenoid content has been established in tomato and, to a lesser extent, pepper. Mature tomato fruits exhibit a wide range of colors, and many mutations and QTL affecting fruit color have been characterized and mapped. Two fruit color mutants in tomato, Del (red to orange) and r (red to yellow), and one mutant in pepper, Y (red to yellow), control qualitative fruit color shifts and have been shown to be identical or tightly linked to structural genes in the carotenoid pathway. Accordingly, from this disclosure, including the Deposit of this invention, and the knowledge in the art, the skilled person has possession of gene(s) or nucleic acid molecule(s) responsible for color and can introduce those gene(s) or molecule(s) into pepper through genetic transformation. A skilled person, from this disclosure, including the Deposit of this invention, and the knowledge in the art, also has possession of nucleic acid molecule(s) responsible for other herein identified traits and can introduce those gene(s) or molecule(s) into pepper through genetic transformation. Thereby, in addition to breeding methods, genetic transformation can be employed to arrive at embodiments of the invention.

Further aspects of the invention relate to Phytophthora capsici resistance. Phytophthora capsici is a fungal pathogen belonging to the group of Oomycetes. The fungus has a broad host range attacking besides pepper also tomato, eggplant, cucumber, watermelon, pumpkin, squash, cocoa, and macadamia plants. Phytophthora capsici can occur all parts of the plants, causing a wide array of symptoms such as stem and vine blight, wilting or fruit rot. On pepper, infection of the stem near the soil line is common. Stem lesions start as dark, water-soaked areas which become brown to black and result in girdling, wilting and plant death. P. capsici may also cause root rot and foliar blight on pepper. On leaves, small, water soaked lesions expand and turn a light tan colour. White moldy growth may be seen on leaves during wet periods. Rapid blighting of leaves and shoots may occur. Pepper fruit can also be infected through the fruit stalk. Fruit rot appears as dark green, water-soaked areas that become covered with a white to gray mold. Infected fruit dries, becomes shrunken and wrinkled, and remains attached to the stem as described on the website of the Ministry of Agriculture of British Columbia under pest management relating to Phytophthora Blight of Cucurbits and Pepper and also in Biles et al., Proc. Okla. Acad. Sci. 75: 1-5,1995.

A skilled person, from this disclosure, including the Deposit of this invention, and the knowledge in the art, also has possession of nucleic acid molecule(s) responsible for other herein identified traits, such as resistance to tobacco mosaic virus (also known as Tobamo virus or TMV and can introduce those gene(s) or molecule(s)associated with these traits into pepper through genetic transformation and breeding selection to arrive at embodiments of the invention.

Genetic transformation may therefore be used to insert a selected transgene into the plant of the invention, being a plant of hybrid pepper variety 10BR46100 or may, alternatively, be used for the preparation of transgenes which can be introduced by backcrossing. Methods for the transformation of plants, including pepper, are well known to those of skill in the art.

Vectors used for the transformation of pepper cells are not limited so long as the vector can express an inserted DNA in the cells. For example, vectors comprising promoters for constitutive gene expression in pepper cells (e.g., cauliflower mosaic virus 35S promoter) and promoters inducible by exogenous stimuli can be used. Examples of suitable vectors include pBI binary vector. The “pepper cell” into which the vector is to be introduced includes various forms of pepper cells, such as cultured cell suspensions, protoplasts, leaf sections, and callus. A vector can be introduced into pepper cells by known methods, such as the polyethylene glycol method, polycation method, electroporation, Agrobacterium-mediated transfer, particle bombardment and direct DNA uptake by protoplasts.

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wound tissues in a controlled manner.

A particularly efficient method for delivering transforming DNA segments to plant cells is microprojectile bombardment. In this method, particles are coated with nucleic acids and delivered into cells by a propelling force. Exemplary particles include those comprised of tungsten, platinum, and preferably, gold. For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate. An illustrative embodiment of a method for delivering DNA into plant cells by acceleration is the Biolistics Particle Delivery System, which can be used to propel particles coated with DNA or cells through a screen, such as a stainless steel or Nytex screen, onto a surface covered with target pepper cells. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large. Microprojectile bombardment techniques are widely applicable, and may be used to transform virtually any plant species, including a plant of hybrid pepper variety 10BR46100.

Agrobacterium-mediated transfer is another widely applicable system for introducing gene loci into plant cells. An advantage of the technique is that DNA can be introduced into whole plant tissues, thereby bypassing the need for regeneration of an intact plant from a protoplast. Agrobacterium transformation vectors are capable of replication in E. coli as well as Agrobacterium, allowing for convenient manipulations. Moreover, advances in vectors for Agrobacterium-mediated gene transfer have improved the arrangement of genes and restriction sites in the vectors to facilitate the construction of vectors capable of expressing various polypeptide coding genes. The vectors have convenient multi-linker regions flanked by a promoter and a polyadenylation site for direct expression of inserted polypeptide coding genes. Additionally, Agrobacterium containing both armed and disarmed Ti genes can be used for transformation. In those plant strains where Agrobacterium-mediated transformation is efficient, it is the method of choice because of the facile and defined nature of the gene locus transfer. The use of Agrobacterium-mediated plant integrating vectors to introduce DNA into plant cells, particularly those belonging to the Solanaeceae family, including pepper plant cells, is well known in the art (See, e.g., U.S. Pats. No. 5,591,616; 7,601,536; 5,939,288; 5,750,386; 5,789,657).

Transformation of plant protoplasts also can be achieved using methods based on calcium phosphate precipitation, polyethylene glycol treatment, electroporation, and combinations of these treatments.

A number of promoters have utility for plant gene expression for any gene of interest including but not limited to selectable markers, scoreable markers, genes for pest tolerance, disease resistance, nutritional enhancements and any other gene of agronomic interest. Promoters include but are not limited to Inducible promoters, Constitutive promoters, Tissue-specific or Tissue-preferred promoters. These are further described in U.S. Patent Application Publication 2009/0313713, the entire contents and disclosure of which are hereby incorporated by reference. Examples of constitutive promoters useful for plant gene expression relating to pepper plants include, but are not limited to, the cauliflower mosaic virus (CaMV) P-35S promoter, a tandemly duplicated version of the CaMV 35S promoter, the enhanced 35S promoter (P-e35S), the nopaline synthase promoter, the octopine synthase promoter, the figwort mosaic virus (P-FMV) promoter (see U.S. Pat. No. 5,378,619), an enhanced version of the FMV promoter (P-eFMV) where the promoter sequence of P-FMV is duplicated in tandem, the cauliflower mosaic virus 19S promoter, a sugarcane bacilliform virus promoter, a commelina yellow mottle virus promoter, the promoter for the thylakoid membrane proteins from pepper (psaD, psaF, psaE, PC, FNR, atpC, atpD, cab, rbcS) (see U.S. Pat. No. 7,161,061), the CAB-1 promoter has been characterized in transgenic tobacco as described in Gotor et al. Plant J. 1993 Apr;3(4):509-18. Analysis of three tissue-specific elements from the wheat Cab-1 enhancer), the promoter from maize prolamin seed storage protein (see U.S. Pat. No. 7,119,255), and other plant DNA virus promoters known to express in plant cells. A variety of plant gene promoters that are regulated in response to environmental, hormonal, chemical, and/or developmental signals can be used for expression of an operably linked gene in plant cells, including promoters regulated by (1) heat, (2) light (e.g., pea rbcS-3A promoter, maize rbcS promoter, or chlorophyll a/b-binding protein promoter), (3) hormones, such as abscisic acid, (4) wounding (e.g., wunl, or (5) chemicals such as methyl jasmonate, salicylic acid, or Safener. It may also be advantageous to employ organ-specific promoters. The use of pepper specific promoters and promoters associated with Capsicum family specific genes are well known in the art (See, e.g., U.S. Pats. No. 6,437,221; 5,945,580; 6,956,147; 7,595,433; 7,501,557)

Exemplary nucleic acids which may be introduced to pepper plants of the invention include, for example, DNA sequences or genes from another species, or even genes or sequences which originate with or are present in pepper species, but are incorporated into recipient cells by genetic engineering methods rather than classical reproduction or breeding techniques. However, the term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the plant cell, DNA from another plant, DNA from a different organism, or a DNA generated externally, such as a DNA sequence containing an antisense message of a gene, or a DNA sequence encoding a synthetic or modified version of a gene.

Many hundreds if not thousands of different genes are known and could potentially be introduced into a plant of hybrid pepper variety 10BR46100. Non-limiting examples of particular genes and corresponding phenotypes one may choose to introduce into a pepper plant include one or more genes for insect tolerance, pest tolerance such as genes for fungal disease control, herbicide tolerance, and genes for quality improvements such as yield, nutritional enhancements, environmental or stress tolerances, or any desirable changes in plant physiology, growth, development, morphology or plant product(s).

Alternatively, the DNA coding sequences can affect these phenotypes by encoding a non-translatable RNA molecule that causes the targeted inhibition of expression of an endogenous gene, for example via antisense- or cosuppression-mediated mechanisms. The RNA could also be a catalytic RNA molecule (i.e., a ribozyme) engineered to cleave a desired endogenous mRNA product. Thus, any gene which produces a protein or mRNA which expresses a phenotype or morphology change of interest is useful for the practice of the present invention. (See also U.S. Pat No. 7,576,262, “Modified gene-silencing RNA and uses thereof.”

U.S. Pats. Nos. 7,230,158, 7,122,720, 7,081,363, 6,734,341, 6,503,732, 6,392,121, 6,087,560, 5,981,181, 5,977,060, 5,608,146, 5,516,667, each of which, and all documents cited therein are hereby incorporated herein by reference, consistent with the above INCORPORATION BY REFERENCE section, are additionally cited as examples of U.S. Patents that relate to general promoters and plant gene expression and may concern transformed pepper and/or methods of transforming pepper or pepper plant cells, and techniques from these U.S. Patents, as well as promoters, vectors, etc., may be employed in the practice of this invention to introduce exogenous nucleic acid sequence(s) into a plant of hybrid pepper variety 10BR46100 (or cells thereof), and exemplify some exogenous nucleic acid sequence(s) which can be introduced into a plant of hybrid pepper variety 10BR46100 (or cells thereof) of the invention, as well as techniques, promoters, vectors etc., to thereby obtain further plants of hybrid pepper variety 10BR46100, plant parts and cells, seeds, other propagation material harvestable parts of these plants, etc. of the invention, e.g. tissue culture, including a cell or protoplast, such as an embryo, meristem, cotyledon, pollen, leaf, anther, root, root tip, pistil, flower, seed or stalk.

The invention further relates to propagation material for producing plants of the invention. Such propagation material comprises inter alia seeds of the claimed plant and parts of the plant that are suitable for sexual reproduction. Such parts are for example selected from the group consisting of seeds, microspores, pollen, ovaries, ovules, embryo sacs and egg cells. In addition, the invention relates to propagation material comprising parts of the plant that are suitable for vegetative reproduction, for example cuttings, roots, stems, cells, protoplasts.

According to a further aspect thereof the propagation material of the invention comprises a tissue culture of the claimed plant. The tissue culture comprises regenerable cells. Such tissue culture can be derived from leaves, pollen, embryos, cotyledon, hypocotyls, meristematic cells, roots, root tips, anthers, flowers, seeds and stems. Tissue culture methodologies relating to pepper plants are well known in the art(See generally U.S. Pat Nos. 7,642,423 and 7,696,416). In vitro regeneration of Solanaceae cultivars is further described in Schuelter A R et al. Genet Mol Res. 2009 August 11;8(3):963-75, In vitro regeneration of cocona (Solanum sessiliflorum, Solanaceae) cultivars for commercial production. In vitro flowering and fruiting in the Capsicum family is described in Brent and Galletta, HORTSCIENCE 30(1):130-132. 1995,In Vitro Flowering and Fruiting of Capsicum fruitescens L. Further aspects of in vitro propagation of pepper plant related families are described in Zelcer et al. Plant Cell Reports, Volume 2, Number 5, 252-254, Shoot regeneration in root cultures of Solanaceae; S. Shrivastava, P. K. Dubey, International Journal of Biotechnology & Biochemistry, January 2007, In-vitro callus induction and shoot regeneration in Withania somnifera Dunal; Sanatombi K., G. J. Sharma, Not. Bot. Hort. Agrobot. Cluj, 2007 Volume 35, Issue 1, MICROPROPAGATION OF CAPSICUM ANNUUM L.; Prakash A H et al. J. Biosci., Vol. 22, Number 3, June 1997, pp 339-344, Plant regeneration from protoplasts of Capsicum annuum L. and Agrawal et al. Plant Cell, Tissue and Organ Culture Volume 16, Number 1, 47-55, Plant regeneration in tissue cultures of pepper (Capsicum annuum L. cv. Mathania).

Also, the invention comprehends methods for producing a seed of a “10BR46100”-derived pepper plant comprising (a) crossing a plant of hybrid pepper variety 10BR46100, representative seed of which having been deposited under NCIMB Accession No. NCIMB 41824, with a second pepper plant, and (b) whereby seed of a “10BR46100”-derived pepper plant form (e.g., by allowing the plant from the cross to grow to producing seed). Such a method can further comprise (c) crossing a plant grown from “10BR46100”-derived pepper seed with itself or with a second pepper plant to yield additional “10BR46100”-derived pepper seed, (d) growing the additional “10BR46100”-derived pepper seed of step (c) to yield additional “10BR46100”-derived pepper plants, and (e) repeating the crossing and growing of steps (c) and (d) to generate further “10BR46100”-derived pepper plants.

The invention further involves a method of determining the genotype of a plant of hybrid pepper variety 10BR46100, representative seed of which has been deposited under NCIMB Accession No. NCIMB 41824, or a first generation progeny thereof, comprising obtaining a sample of nucleic acids from said plant and detecting in said nucleic acids a plurality of polymorphisms. This method can additionally comprise the step of storing the results of detecting the plurality of polymorphisms on a computer readable medium and/or transmitting the results of detecting the plurality of polymorphisms, e.g., by telephony or by means of computer (e.g., via email). The plurality of polymorphisms are indicative of and/or give rise to the expression of the morphological and physiological characteristics of hybrid pepper variety 10BR46100.

Mention is again made of U.S. Patent Publication 2009/0313713, incorporated herein by reference, because peppers having the herein identified traits as well as glucose and/or fructose content as in that publication are contemplated by this invention, e.g., through crossing plants having herein identified traits with those of U.S. Patent Publication 2009/0313713 to arrive at plants having the herein identified traits and those of U.S. Patent Publication 2009/0313713; and, with regard to expression vectors for pepper transformation, marker genes, promoters (including inducible, constitutive and tissue specific or preferred promoters), signal sequences for targeting proteins to subcellular compartments, foreign protein genes and agronomic genes, genes that confer resistance to pests or disease or herbicide, methods for transformation, including Agrobacterium-Mediated Transformation, Direct Gene Transfer, Single Gene Transformations, Tissue Culture reproduction and regeneration, and additional breeding methods, all of which can also be employed in the practice of the present invention.

The invention is further described by the following numbered paragraphs:

1. Pepper plant exhibiting a combination of traits including red colored blocky fruits, Tobamovirus resistance against Tobamovirus Pathotype 0 (P0) and Tobamovirus Pathotype 1-2 (P1-2), and resistance against Phytophthora capsici, representative seed of which having been deposited under NCIMB Accession No. 41824.

2. Pepper plant designated 10BR46100, representative seed of which have deposited under NCIMB Accession No. 41824.

3. A pepper plant having all the morphological and physiological characteristics of a plant as in paragraph 2, representative seed of which have deposited under NCIMB Accession No. 41824.

4. Seed of the plant of paragraph 1.

5. Parts of the plant of paragraph 1, wherein said parts of the plant are suitable for sexual reproduction.

6. Parts of the plant as in paragraph 5, said parts selected from the group consisting of microspores, pollen, ovaries, ovules, embryo sacs and egg cells.

7. Parts of the plant of paragraph 1, wherein said parts of the plant are suitable for vegetative reproduction.

8. Parts as in paragraph 7, said parts selected from the group consisting of cuttings, roots, stems, cells and protoplasts.

9. A tissue culture of regenerable cells from the pepper plant of paragraph 1.

10. A tissue culture as in paragraph 8, wherein said cells or protoplasts of the tissue culture which are derived from a tissue selected from the group consisting of leaves, pollen, embryos, cotyledon, hypocotyls, meristematic cells, roots, root tips, anthers, flowers, seeds and stems.

11. Progeny of a pepper plant of paragraph 1.

12. Progeny as in paragraph 11, wherein said progeny is produced by sexual or vegetative reproduction of said pepper plant.

13. Progeny of a pepper plant of paragraph 1, wherein the plant exhibits a combination of traits including red colored blocky fruits, Tobamovirus resistance against Tobamovirus Pathotype 0 (P0) and Tobamovirus Pathotype 1-2 (P1-2), and resistance against Phytophthora capsici, as in hybrid pepper variety 10BR46100, representative seed of which have deposited under NCIMB Accession No. 41824, and is modified in one or more other characteristics.

14. Progeny as in paragraph 13, wherein the modification is effected by mutagenesis.

15. Progeny as in paragraph 13, wherein the modification is effected by transformation with a transgene.

16. A method of producing an inbred pepper plant derived from hybrid pepper variety 10BR46100, comprising the steps:

a) preparing a progeny plant derived from hybrid pepper variety 10BR46100 by crossing the plant of paragraph 1 with a second pepper plant;

b) crossing the progeny plant with itself or a second pepper plant to produce a seed of a progeny plant of a subsequent generation;

c) growing a progeny plant of a subsequent generation from said seed and crossing the progeny plant of a subsequent generation with itself or a second pepper plant; and

d) repeating step b) or c) for at least 1 more generation to produce an inbred pepper plant derived from the hybrid pepper variety 10BR46100.

17. A method of determining the genotype of a plant of hybrid pepper variety 10BR46100, representative seed of which has been deposited under NCIMB Accession No. NCIMB 41824, or a first generation progeny thereof, comprising obtaining a sample of nucleic acids from said plant and detecting in said nucleic acids a plurality of polymorphisms, wherein the plurality of polymorphisms are indicative of and/or give rise to the expression of the morphological and physiological characteristics of inbred hybrid pepper variety 10BR46100.

18. The method of paragraph 17 additionally comprising the step of storing the results of detecting the plurality of polymorphisms on a computer readable medium, or transmitting the results of detecting the plurality of polymorphisms.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. Pepper plant exhibiting a combination of traits including red colored blocky fruits, Tobamovirus resistance against Tobamovirus Pathotype 0 (P0) and Tobamovirus Pathotype 1-2 (P1-2), and resistance against Phytophthora capsici, representative seed of which having been deposited under NCIMB Accession No. 41824.

2. Pepper plant designated 10BR46100, representative seed of which have deposited under NCIMB Accession No. 41824.

3. A pepper plant having all the morphological and physiological characteristics of a plant as claimed in claim 2, representative seed of which have deposited under NCIMB Accession No. 41824.

4. Seed of the plant of claim 1.

5. Parts of the plant of claim 1, wherein said parts of the plant are suitable for sexual reproduction.

6. Parts of the plant as claimed in claim 5, said parts selected from the group consisting of microspores, pollen, ovaries, ovules, embryo sacs and egg cells.

7. Parts of the plant of claim 1, wherein said parts of the plant are suitable for vegetative reproduction.

8. Parts as claimed in claim 7, said parts selected from the group consisting of cuttings, roots, stems, cells and protoplasts.

9. A tissue culture of regenerable cells from the pepper plant of claim 1.

10. A tissue culture as claimed in claim 8, wherein said cells or protoplasts of the tissue culture which are derived from a tissue selected from the group consisting of leaves, pollen, embryos, cotyledon, hypocotyls, meristematic cells, roots, root tips, anthers, flowers, seeds and stems.

11. Progeny of a pepper plant of claim 1.

12. Progeny as claimed in claim 11, wherein said progeny is produced by sexual or vegetative reproduction of said pepper plant.

13. Progeny of a pepper plant of claim 1, wherein the plant exhibits a combination of traits including red colored blocky fruits, Tobamovirus resistance against Tobamovirus Pathotype 0 (P0) and Tobamovirus Pathotype 1-2 (P1-2), and resistance against Phytophthora capsici, as in hybrid pepper variety 10BR46100, representative seed of which have deposited under NCIMB Accession No. 41824, and is modified in one or more other characteristics.

14. Progeny as claimed in claim 13, wherein the modification is effected by mutagenesis.

15. Progeny as claimed in claim 13, wherein the modification is effected by transformation with a transgene.

16. A method of producing an inbred pepper plant derived from hybrid pepper variety 10BR46100, comprising the steps:
a) preparing a progeny plant derived from hybrid pepper variety 10BR46100 by crossing the plant of claim 1 with a second pepper plant;
b) crossing the progeny plant with itself or a second pepper plant to produce a seed of a progeny plant of a subsequent generation;
c) growing a progeny plant of a subsequent generation from said seed and crossing the progeny plant of a subsequent generation with itself or a second pepper plant; and
d) repeating step b) or c) for at least 1 more generation to produce an inbred pepper plant derived from the hybrid pepper variety 10BR46100.

17. A method of determining the genotype of a plant of hybrid pepper variety 10BR46100, representative seed of which has been deposited under NCIMB Accession No. NCIMB 41824, or a first generation progeny thereof, comprising obtaining a sample of nucleic acids from said plant and detecting in said nucleic acids a plurality of polymorphisms, wherein the plurality of polymorphisms are indicative of and/or give rise to the expression of the morphological and physiological characteristics of inbred hybrid pepper variety 10BR46100.

18. The method of claim 17 additionally comprising the step of storing the results of detecting the plurality of polymorphisms on a computer readable medium, or transmitting the results of detecting the plurality of polymorphisms.
